(19) United States
(12) Patent Application Publication
(10) Pub. No.: US 2005/0070513 A1
(43) Pub. Date: Mar. 31, 2005

(54) Treatment of inflammatory bowel disease with vitamin D compounds

(75) Inventor: Margherita T. Cantorna, State College, PA (US)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: The Penn State Research Foundation, University Park, PA (US)

(21) Appl. No.: 10/969,394

(22) Filed: Oct. 20, 2004

Related U.S. Application Data
(63) Continuation of application No. 10/148,620, filed on Aug. 19, 2002, now abandoned.
(60) Provisional application No. 60/168,501, filed on Dec. 2, 1999.
(60) Provisional application No. 60/197,827, filed on Apr. 14, 2000.
(60) Provisional application No. 60/208,632, filed on Jun. 1, 2000.
(60) Provisional application No. 60/231,906, filed on Sep. 11, 2000.

(51) Int. Cl. A61K 31/59

(52) U.S. Cl. 514/167

(57) ABSTRACT

A method of treating inflammatory bowel disease, particularly ulcerative colitis and Crohn's disease, is disclosed. The method involves administering a vitamin D compound in an amount effective to treat the disease. The administration of a vitamin D compound also prevents the development of or delays the onset of inflammatory bowel disease in susceptible individuals.

### BACKGROUND OF THE INVENTION

This invention relates to vitamin D compounds, and more particularly to the use of vitamin D compounds to treat inflammatory bowel disease.

The natural hormone, 1α,25-dihydroxyvitamin D₃ and its analog 1α,25-dihydroxyvitamin D₂ are known to be highly potent regulators of calcium homeostasis in animals and humans, and their activity in cellular differentiation has also been established, Ostrem et al., Proc. Natl. Acad. Sci. USA, 84, 2610 (1987). Many structural analogs of these metabolites have been prepared and tested, including 1α-hydroxyvitamin D₃, 1α-hydroxyvitamin D₂, various side chain homologated vitamins and fluorinated analogs. Some of these compounds exhibit an interesting separation of activities in cell differentiation and calcium regulation. This difference in activity may be useful in the treatment of a variety of diseases such as renal osteodystrophy, vitamin D-resistant rickets, osteoporosis, psoriasis, and certain malignancies.

Inflammatory bowel diseases (IBD) are immune mediated diseases of unknown etiology affecting the gastrointestinal (GI) tract. There are at least two distinct forms of IBD, ulcerative colitis and Crohn's disease. IBD are chronic recurring illnesses most commonly involving inflammation of the terminal ileum and colon, although these diseases can also affect many sites throughout the alimentary tract. Clearly, genetic factors predispose individuals to development of IBD (Podolosky 1991). In addition, the environment contributes to IBD development, and there is reason to believe that vitamin D may be an environmental factor which affects IBD. Vitamin D from sunlight exposure is less in areas where IBD occurs most often, as IBD is most prevalent in northern climates such as North America and Northern Europe (Podolosky 1991, Sonnenberg et al. 1991). A major source of vitamin D results from its manufacture via a photolysis reaction in the skin, and vitamin D available from sunlight exposure is significantly less in northern climates, and especially low during the winter (Clemens et al. 1982, DeLuca 1993). Dietary intake of vitamin D is problematic since there are few foods which are naturally rich in vitamin D. Weight loss occurs in 65-75% of patients diagnosed with Crohn's disease and 18-62% of patients with ulcerative colitis (Fleming 1995, Geerling et al. 1998). Vitamin deficiencies in general and vitamin D deficiency in particular have been shown to occur in IBD patients (Andreassen et al. 1998, Kuroki et al. 1993). To date the possible association between vitamin D status and the incidence and severity of IBD in humans or animals has not been studied. The anecdotal information suggests that vitamin D status could be an environmental factor affecting the prevalence rate for IBD and that the correlation warrants investigation.

The identification of vitamin D receptors in peripheral blood mononuclear cells sparked the early interest in vitamin D as an immune system regulator (Bhalla et al. 1983, Provvedini et al. 1983). In particular the CD4+ Th cells have vitamin D receptors and are therefore targets for vitamin D (Veldman et al. 2000). Hormonally active vitamin D (1,25-dihydroxycholecalciferol) suppressed the development of at least two experimental autoimmune diseases (Cantorna et al. 1996, Cantorna et al. 1998a). In vitro 1,25-dihydroxycholecalciferol inhibited T cell proliferation and decreased the production of interleukin (IL)-2, interferon (IFN)-γ, and tumor necrosis factor (TNF)-α (Lemire 1992a). In vivo 1,25-dihydroxycholecalciferol injections were shown to inhibit the delayed type hypersensitivity reaction associated with the type-1 helper T (Th1) cell response (Lemire et al. 1991, Lemire et al. 1992b). Vitamin D is a potent regulator of the immune system in general and T cells specifically.

For IBD, the immune mediated attack is against the GI tract (Niessner and Volk 1995, Podolosky 1991). T cells, which preferentially produce the Th1 cytokines (IL-2, IFN-γ, and TNF-α), have been shown to transfer Crohn's-like symptoms to naive mice (Aranda et al. 1997, Bregenholt and Claesson 1998) and the production of Th1 cytokines is associated with IBD in humans as well (Niessner and Volk 1995). 1,25-dihydroxycholecalciferol treatment has been shown to suppress the development of other T cell mediated experimental autoimmune diseases (multiple sclerosis, and arthritis; Cantorna et al. 1996, Cantorna et al. 1998a). The hypothesis that vitamin D (through the production of 1,25-dihydroxycholecalciferol) would suppress the development and progression of IBD thus seemed credible.

Standard treatments of patients with IBD include short-term high dose and long term low dose prednisone use (Podolosky 1991, Andreassen et al. 1998). Prednisone and other corticosteroid therapies result in a decreased bone mineral density and many times result in higher risks for vertebral fracture (Andreassen et al. 1997, Andreassen et al. 1998). Vitamin D supplementation of patients on corticosteroids has been shown to prevent steroid induced bone loss (Buckley et al. 1996). The hormonally active form of vitamin D (1,25-dihydroxycholecalciferol) is known to increase bone mineralization when given to experimental animals (Cantora et al. 1998b) and people (Ongphiphadhanakul et al. 2000). Thus a further benefit of vitamin D and or 1,25-dihydroxycholecalciferol supplementation may be the maintenance of bone mineral density.

### SUMMARY OF THE INVENTION

The present invention is a method of preventing inflammatory bowel diseases (IBD) in susceptible individuals and treating patients with IBD by administering an amount of a vitamin D compound, preferably 1,25(OH)₂D₃ or analogs thereof, effective to prevent IBD development or to diminish IBD symptoms, respectively. The method comprises selecting an IBD patient and administering a sufficient amount of the vitamin D analog to the patient such that the IBD symptoms are abated.

Structurally the vitamin D compounds found useful to treat IBD are characterized by the general formula I shown below: 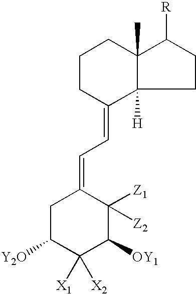
where Y₁ and Y₂, which may be the same or different, are each selected from the group consisting of hydrogen and a hydroxy-protecting group, where Z₁ and Z₂ are both hydrogen or Z₁ and Z₂ together are ═CH₂, where X₁ and X₂ are both hydrogen, or one is hydrogen and the other is O-aryl, O-alkyl, alkyl, hydroxyalkyl or fluoroalkyl and can have an α or β configuration, or taken together represent an alkylidene group ═CR₆R₇ where R₆ and R₇, which may be the same or different, are each selected from the group consisting of hydrogen, alkyl, hydroxyalkyl and fluoroalkyl, or, when taken together represent the group —(CH₂)ₓ— where X is an integer from 2 to 5, and can have an α or β configuration, and where the group R represents any of the typical side chains known for vitamin D type compounds.

More specifically R can represent a saturated or unsaturated hydrocarbon radical of 1 to 35 carbons, that may be straight-chain, branched or cyclic and that may contain one or more additional substituents, such as hydroxy- or protected-hydroxy groups, fluoro, carbonyl, ester, epoxy, amino or other heteroatomic groups. Preferred side chains of this type are represented by the structure below 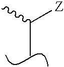
where the stereochemical center (corresponding to C-20 in steroid numbering) may have the R or S configuration, (i.e. either the natural configuration about carbon 20 or the 20-epi configuration), and where Z is selected from Y, —OY, —CH₂OY, —C≡CY and —CH═CHY, where the double bond may have the cis or trans geometry, and where Y is selected from hydrogen, methyl, —COR⁵ and a radical of the structure: 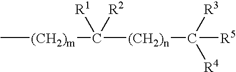
where m and n, independently, represent the integers from 0 to 5, where R¹ is selected from hydrogen, deuterium, hydroxy, protected hydroxy, fluoro, trifluoromethyl, and C₁₋₅-alkyl, which may be straight chain or branched and, optionally, bear a hydroxy or protected-hydroxy substituent, and where each of R², R³, and R⁴, independently, is selected from deuterium, deuteroalkyl, hydrogen, fluoro, trifluoromethyl and C₁₋₅ alkyl, which may be straight-chain or branched, and optionally, bear a hydroxy or protected-hydroxy substituent, and where R¹ and R², taken together, represent an oxo group, or an alkylidene group, ═CR²R³, or the group —(CH₂)ₚ—, where p is an integer from 2 to 5, and where R³ and R⁴, taken together, represent an oxo group, or the group —(CH₂)_{q}—, where q is an integer from 2 to 5, and where R⁵ represents hydrogen, hydroxy, protected hydroxy, or C₁₋₅ alkyl and wherein any of the CH-groups at positions 20, 22, or 23 in the side chain may be replaced by a nitrogen atom, or where any of the groups —CH(CH₃)—, —CH(R³)—, or —CH(R²)— at positions 20, 22, and 23, respectively, may be replaced by an oxygen or sulfur atom.

The wavy line to the methyl substituent at C-20 indicates that carbon 20 may have either the R or S configuration.

Specific important examples of side chains with natural 20R-configuration are the structures represented by formulas (a), b), (c), (d) and (e) below. i.e. the side chain as it occurs in 25-hydroxyvitamin D₃ (a); vitamin D₃ (b); 25-hydroxyvitamin D₂ (c); vitamin D₂ (d); and the C-24 epimer of 25-hydroxyvitamin D₂ (e):

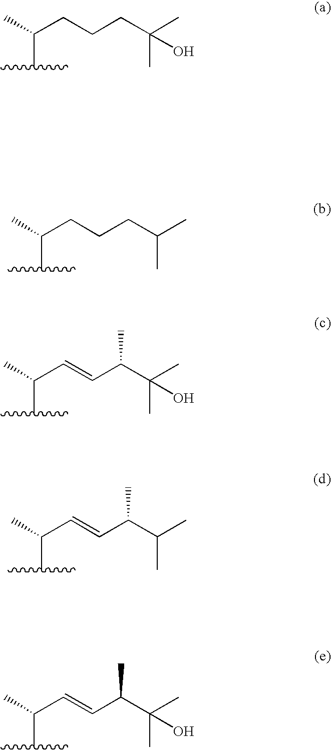

Vitamin D analogs such as but not limited to the following are particularly preferred: 1,25-dihydroxyvitamin D₃; 1α-hydroxyvitamin D₃; 1,25-dihydroxyvitamin D₂; 19-nor-1,25-dihydroxyvitamin D₂; 26,27-hexafluoro-1,25-dihydroxyvitamin D₂; 1,25-dihydroxy-24(E)-dehydro-24-homo-vitamin D₃; 19-nor-1,25-dihydroxy-21-epi-vitamin D₃; 1α,25 dihydroxyvitamin D₃ triacetate; and 25-acetyl-1α,25 dihydroxyvitamin D₃. In a most preferred form of the invention, the compound is 1,25(OH)₂D₃.

The above compounds may be present in a composition to treat IBD in an amount from about 0.01 μg/gm to about 100 μg/gm of the composition, and may be administered topically, transdermally, orally or parenterally in dosages of from about 0.01 μg/day to about 100 μg/day.

A preferred dose of vitamin D compound for the present invention is the maximum that a patient can tolerate and not develop serious hypercalcemia. If the vitamin D compound is not a 1α-hydroxy compound, a particularly advantageous daily dose of vitamin D compound is between 5.0 and 50 μg per day per 160 pound patient If the vitamin D compound is a 1α-hydroxy compound, the preferred dose is between 0.5 and 10 μg per day per 160 pound patient. If the patient has calcium intakes of above 800 mg/day, doses of 1,25(OH)₂D₃ over 0.75 μg per day per 160 pound patient are not preferred. If the patient is on a low calcium diet and/or takes the dose late at night, higher doses of 1,25(OH)₂D₃ would be possible and would be preferred. In this embodiment of the invention, the amount of 1,25(OH)₂D₃ administered could be as high as 1.5 μg per day per 160 pound patient. A preferred dose of 1,25(OH)₂D₃ would be 0.5-1.0 μg per day per 160 pound patient.

### BRIEF DESCRIPTION OF THE DRAWINGS

FIG. 1 is a graph illustrating the mortality rate of interleukin-10 knockout mice (IL-10 KO) either maintained deficient of 1,25(OH)₂D₃ (−D) or supplemented with 1,25(OH)₂D₃ (+D) as compared to interleukin-10 wildtype mice (IL-10 Wt) deficient of 1,25(OH)₂D₃; and

FIG. 2 is a graph illustrating growth curves for IL-10 KO mice either maintained deficient of 1,25(OH)₂D₃ (−D) or supplemented with 1,25(OH)₂D₃ (+D) as compared to IL-10 Wt mice deficient of 1,25(OH)₂D₃.

### DETAILED DESCRIPTION OF THE INVENTION

As used in the description and in the claims, the term “hydroxy-protecting group’ signifies any group commonly used for the temporary protection of hydroxy functions, such as for example, alkoxycarbonyl, acyl, alkylsilyl or alkylarylsilyl groups (hereinafter referred to simply as “silyl” groups), and alkoxyalkyl groups. Alkoxycarbonyl protecting groups are alkyl-O—CO— groupings such as methoxycarbonyl, ethoxycarbonyl, propoxycarbonyl, isopropoxycarbonyl, butoxycarbonyl, isobutoxycarbonyl, tert-butoxycarbonyl, benzyloxycarbonyl or allyloxycarbonyl. The term “acyl” signifies an alkanoyl group of 1 to 6 carbons, in all of its isomeric forms, or a carboxyalkanoyl group of 1 to 6 carbons, such as an oxalyl, malonyl, succinyl, glutaryl group, or an aromatic acyl group such as benzoyl, or a halo, nitro or alkyl substituted benzoyl group. The word “alky” as used in the description or the claims, denotes a straight-chain or branched alkyl radical of 1 to 10 carbons, in all its isomeric forms. Alkoxyalkyl protecting groups are groupings such as methoxymethyl, ethoxymethyl, methoxyethoxymethyl, or tetrahydrofuranyl and tetrahydropyranyl. Preferred silyl-protecting groups are trimethylsilyl, triethylsilyl, t-butyldimethylsilyl, dibutylmethylsilyl, diphenylmethylsilyl, phenyldimethylsilyl, diphenyl-t-butylsilyl and analogous alkylated silyl radicals. The term “aryl” specifies a phenyl-, or an alkyl-, nitro- or halo-substituted phenyl group.

A “protected hydroxy” group is a hydroxy group derivatised or protected by any of the above groups commonly used for the temporary or permanent protection of hydroxy functions, e.g. the silyl, alkoxyalkyl, acyl or alkoxycarbonyl groups, as previously defined. The terms “hydroxyalkyl”, “deuteroalkyl” and “fluoroalkyl” refer to an alkyl radical substituted by one or more hydroxy, deuterium or fluoro groups respectively.

The term “vitamin D compound” refers to the compounds defined by general formula I. It should be noted in this description that the term “24-homo” refers to the addition of one methylene group and the term “24-dihomo” refers to the addition of two methylene groups at the carbon 24 position in the side chain. Likewise, the term “trihomo” refers to the addition of three methylene groups. Also, the term “26,27-dimethyl” refers to the addition of a methyl group at the carbon 26 and 27 positions so that for example R³ and R⁴ are ethyl groups. Likewise, the term “26,27-diethyl” refers to the addition of an ethyl group at the 26 and 27 positions so that R³ and R⁴ are propyl groups.

In the following lists of compounds, if an alkylidene or alkyl substituent is attached at the carbon 2 position then the particular alkylidene or alkyl substituent should be added to the nomenclature. For example, if a methylene group is the alkylidene substituent, the term “2-methylene” should proceed each of the named compounds. If an ethylene group is the alkylidene substituent, the term “2-ethylene” should proceed each of the named compounds, and so on. Likewise, if a methyl or ethyl group is the alkyl substituent, then the term “2-methyl” or “2-ethyl” respectively should proceed each of the named compounds, and so on. Also, if Z₁ and Z₂ in formula I are both hydrogens, then the term “19-nor” should proceed each of the named compounds. In addition, if the methyl group attached at the carbon 20 position is in its epi or unnatural configuration, the term “20(S)” or “20-epi” should be included in each of the following named compounds. The named compounds could also be of the vitamin D₂ and/or D₄ type if desired.

Specific and preferred examples of the vitamin D compounds of structure I when the side chain is unsaturated are:
1α-hydroxy-22-dehydrovitamin D₃;
1,25-dihydroxy-22-dehydrovitamin D₃;
1,24-dihydroxy-22-dehydrovitamin D₃;
24-homo-1,25-dihydroxy-22-dehydrovitamin D₃;
24-dihomo-1,25-dihydroxy-22-dehydrovitamin D₃;
24-trihomo-1,25-dihydroxy-22-dehydrovitamin D₃;
26,27-dimethyl-24-homo-1,25-dihydroxy-22-dehydrovitamin D₃;
26,27-dimethyl-24-dihomo-1,25-dihydroxy-22-dehydrovitamin D₃;
26,27-dimethyl-24-trihomo-1,25-dihydroxy-22-dehydrovitamin D₃;
26,27-diethyl-24-homo-1,25-dihydroxy-22-dehydrovitamin D₃;
26,27-diethyl-24-dihomo-1,25-dihydroxy-22-dehydrovitamin D₃;
26,27-diethyl-24-trihomo-1,25-dihydroxy-22-dehydrovitamin D₃;
26,27-dipropyl-24-homo-1,25-dihydroxy-22-dehydrovitamin D₃;
26,27-dipropyl-24-dihomo-1,25-dihydroxy-22-dehydrovitamin D₃; and
26,27-dipropyl-24-trihomo-1,25-dihydroxy-22-dehydrovitamin D₃.

Specific and preferred examples of the vitamin D compounds of structure I when the side chain is saturated are:
1α-hydroxyvitamin D₃;
1,25-dihydroxyvitamin D₃;
1,24-dihydroxyvitamin D₃;
24-homo-1,25-dihydroxyvitamin D₃;
24-dihomo-1,25-dihydroxyvitamin D₃;
24-trihomo-1,25-dihydroxyvitamin D₃;
26,27-dimethyl-24-homo-1,25-dihydroxyvitamin D₃;
26,27-dimethyl-24-dihomo-1,25-dihydroxyvitamin D₃;
26,27-dimethyl-24-trihomo-1,25-dihydroxyvitamin D₃;
26,27-diethyl-24-homo-1,25-dihydroxyvitamin D₃;
26,27-diethyl-24-dihomo-1,25-dihydroxyvitamin D₃;
26,27-diethyl-24-trihomo-1,25-dihydroxyvitamin D₃;
26,27-dipropyl-24-homo-1,25-dihydroxyvitamin D₃;
26,27-dipropyl-24-dihomo-1,25-dihydroxyvitamin D₃; and
26,27-dipropyl-24-trihomo-1,25-dihydroxyvitamin D₃.

The preparation of vitamin D compounds having the basic structure I can be accomplished by a common general method, i.e. the condensation of a bicyclic Windaus-Grundmann type ketone II with the allylic phosphine oxide III to the corresponding vitamin D analogs IV followed by deprotection, if desired, at C-1 and C-3 in the latter compounds: 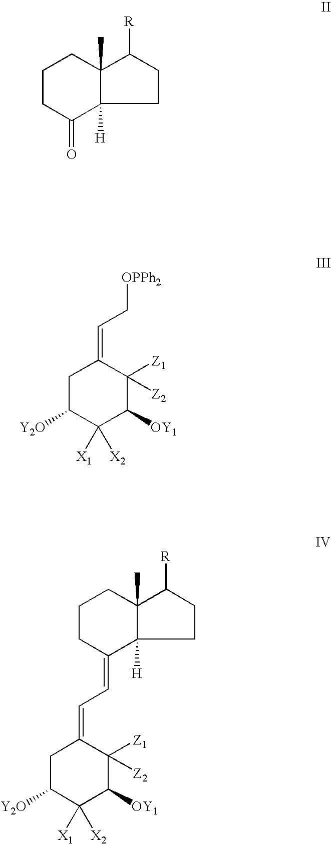
In the structures II, III, and IV groups X₁, and X₂, Y₁ and Y₂, Z₁, and Z₂, and R represent groups defined above; Y₁ and Y₂ are preferably hydroxy-protecting groups, it being also understood that any functionalities in R that might be sensitive, or that interfere with the condensation reaction, be suitably protected as is well-known in the art. The process shown above represents an application of the convergent synthesis concept, which has been applied effectively for the preparation of vitamin D compounds [e.g. Lythgoe et al., J. Chem. Soc. Perkin Trans. I, 590 (1978); Lythgoe, Chem. Soc. Rev. 9, 449 (1983); Toh et al., J. Org. Chem. 48, 1414 (1983); Baggiolini et al., J. Org. Chem. 51, 3098 (1986); Sardina et al., J. Org. Chem. 51, 1264 (1986); J. Org. Chem. 51, 1269 (1986); DeLuca et al., U.S. Pat. No. 5,086,191; DeLuca et al., U.S. Pat. No. 5,536,713].

Hydrindanones of the general structure II are known, or can be prepared by known methods. Specific important examples of such known bicyclic ketones are the structures with the side chains (a), (b), (c) and (d) described above, i.e. 25-hydroxy Grundmann's ketone (f) [Baggiolini et al., J. Org. Chem, 51, 3098 (1986)]; Grundmann's ketone (g) [Inhoffen et al., Chem. Ber. 90, 664 (1957)]; 25-hydroxy Windaus ketone (h) [Baggiolini et al., J. Org. Chem., 51, 3098 (1986)] and Windaus ketone (i) [Windaus et al., Ann., 524, 297 (1936)]:

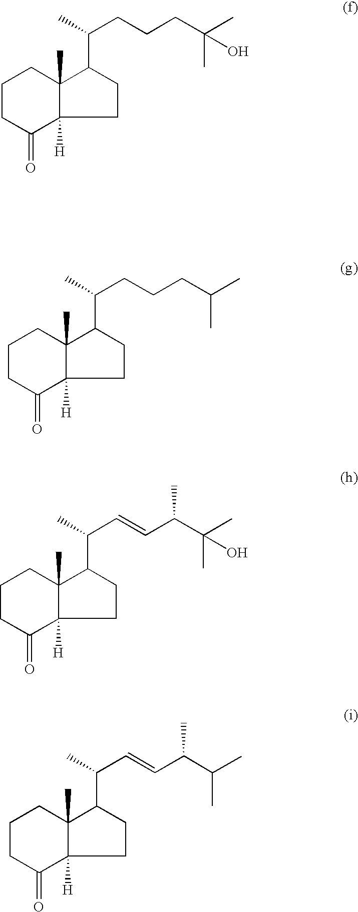

For the preparation of the required phosphine oxides of general structure III, a synthetic route has been developed starting from a methyl quinicate derivative, easily obtained from commercial (1R,3R,4S,5R)-(−)-quinic acid as described by Perlman et al., Tetrahedron Lett. 32, 7663 (1991) and DeLuca et al., U.S. Pat. No. 5,086,191.

The overall process of the synthesis of compounds I is illustrated and described more completely in U.S. Pat. No. 5,945,410 issued Aug. 31, 1999 and entitled “2-Alkyl-19-Nor-Vitamin D Compounds” the specification of which is specifically incorporated herein by reference.

This invention is further described by the following illustrative example. This example demonstrates that vitamin D deficiency exacerbates symptoms of IBD in IL-10 KO mice. Vitamin D deficiency also exacerbated the symptoms of enterocolitis in the animal model. These data predict that both forms of IBD (ulcerative colitis and Chron's disease) are amenable to treatment with 1,25(OH)₂D₃ and other vitamin D analogs.

### EXAMPLE 1

Recently a number of transgenic animals have been developed in which IBD symptoms occur spontaneously. One of the best animal models for Crohn's disease is the IL-10 knockout (KO) mouse (Kuhn et al. 1993, Mac Donald 1994). In conventional animal facilities, the IL-10 KO mice develop enterocolitis within 5-8 weeks of life (Kuhn et al. 1993). Approximately 30% of the IL-10 KO mice die following the development of severe anemia and weight loss (Kuhn et al. 1993). The enterocolitis which develops in IL-10 KO mice is due to an uncontrolled immune response to conventional microflora since germfree IL-10 KO mice do not develop disease. In addition mice raised in specific pathogen free facilities develop milder disease which doesn't result in the death of the mice (Kuhn et al. 1993). There are limitations involved in studying IL-10 KO mice as a model of IBD. If vitamin D is a regulator of IL-10 production then the results in this animal model may not represent what may happen in a “normal” immune response. However patients with Crohn's disease show similar symptoms, have depressed IL-10 production, and have been successfully treated with IL-10 (Narula et al. 1998).

Materials and Methods

MICE. Age and sex matched C57BL/6 IL-10 KO, and wildtype (WT) mice were produced in the Pennsylvania State University breeding colony; the breeding pairs were obtained from Jackson Laboratory (Bar Harbor, Me.). The animal facilities at the Pennsylvania State University are specific pathogen free and therefore breeding IL-10 KO mice was successful. All of the procedures described were reviewed and approved by the Pennsylvania State University Institutional Animal Care and Use Committee on Jan. 25, 1999, IACUC# number: 98118-A0.

DIETS. From a single pool of breeding females fed commercial mouse diet (#5105 Ralston Purina Co.), females in the second week of gestation were selected and randomly distributed into two groups. Starting pregnant dams on vitamin D deficient diet ensured that by 5 weeks of age the weanlings were vitamin D deficient (Cantorna et al. 1996). All mice were fed synthetic diets made in the laboratory (Yang et al. 1993; modification of Smith et al. 1987). The mice were vitamin D deficient, vitamin D sufficient or 1,25-dihydroxycholecalciferol supplemented. Mice were housed under yellow light to prevent the synthesis of vitamin D in skin. All of the mice were vitamin D deficient until weaning.

The 3 week old vitamin D deficient mice were randomly assigned to various treatment groups as described below. Because 1,25(OH)₂D₃ treatment of other experimental autoimmune diseases was most effective when dietary calcium was high (1 g calcium/100 g diet), all mice were fed high calcium diets (Cantorna et al. 1999). Experimental diets were prepared fresh and replaced every 2-3 days during the experiment. To ensure that 1,25-dihydroxycholecalciferol (1,25(OH)₂D₃) treated mice ate all of the 1,25(OH)₂D₃ provided, food cups containing 8 g of diet were replaced every other day (completely eaten) for the duration of each experiment (Cantorna et al. 1996, Cantorna et al. 1998a). In order to monitor vitamin D toxicity, 1,25(OH)₂D₃ supplemented mice were observed daily for signs of hypercalcemia Symptoms of hypercalcemia monitored daily included overall health and weight loss.

VITAMIN D TREATMENTS. In the first experimental design the 3 week old vitamin D deficient mice were either maintained vitamin D deficient or switched to the experimental diet which included 5.0 μg cholecalciferol/d (vitamin D sufficient). The severity of IBD development was compared in vitamin D deficient and vitamin D sufficient mice.

In the 2nd series of experiments 3 week old vitamin D deficient mice were split into 2 groups. One group of mice was maintained on the vitamin D deficient diet and the other group was supplemented with 0.005 μg/d 1,25(OH)₂D₃. The vitamin D deficient and 1,25(OH)₂D₃ supplemented mice were sacrificed 4 weeks later at 9 weeks of age.

In the 3rd experimental design 1,25(OH)₂D₃ treatment was started at the first signs of IBD symptoms (diarrhea, 7 weeks of age). Seven week old vitamin D deficient mice were split into 2 groups. One group was maintained vitamin D deficient and the other group was supplemented with 0.2 μg/d 1,25(OH)₂D₃. The mice were treated for 2 weeks and the 9 week old mice were sacrificed.

FOOD RESTRICTION. Because of the dramatic weight loss and death of vitamin D deficient IL-10 KO mice a series of controlled feeding experiments were done. These experiments used 3 groups of mice. All of the mice for these experiments were maintained vitamin D deficient for the first 5 weeks of life (the earliest signs of weight loss). At 5 weeks the vitamin D deficient IL-10 KO mice were split into 2 groups. Half of the mice were maintained vitamin D deficient and the other half were switched to a vitamin D sufficient diet, which contained 5.0 μg/d cholecalciferol. In addition a group of vitamin D deficient WT mice were also switched to a diet which contained 5.0 μg cholecalciferol/d diet. The food eaten by vitamin D deficient IL-10 KO mice was weighed daily and the vitamin D sufficient IL-10 KO and WT mice were fed a restricted diet which contained the amount of food eaten by vitamin D deficient IL-10 KO mice in the previous 24 hours.

SERUM MEASUREMENTS. Mice were bled at 5 weeks of age and at the end of the experiments to measure hemoglobin, calcium, and red blood cell numbers. Serum was collected every 2 weeks and serum calcium measured (normal for mice is 2.00-2.75 mmol/L). Vitamin D deficiency was also monitored by serum calcium analysis (serum calcium less then 1.75 mmol/L). Calcium (587-A) and hemoglobin (525-A) colorometric kits were from Sigma Chemical Co. (St Louis, Mo.). Red blood cells were counted using a hemocytometer.

IBD SEVERITY. Mortality associated with the development of diarrhea was recorded in IL-10 KO and WT mice. In addition the small intestines (SI) were removed and weighed. The jejunum of the IL-10 KO mice is visibly inflamed and has been used by others to monitor symptoms of IBD in mice (Kuhns et al. 1993). The jejunum of the SI was saved in 100 g/L formalin in phosphate buffered saline solution and sent to the Penn State Diagnostic Laboratory for sectioning and staining with hematoxyalin and eosin. Four or more paraffin sections (4 microns) from each mouse were scored using the exact procedure described by Kuhns et. al (1993). The sections were scored blindly on a scale of 0 to 5 for inflammation. 0-was no inflammation, 1-was a few inflammatory cells, 2-was mild inflammation, 3-was abscess formation, 4-was abscess formation with many inflammatory cells throughout, and 5-was massive inflammation throughout the section.

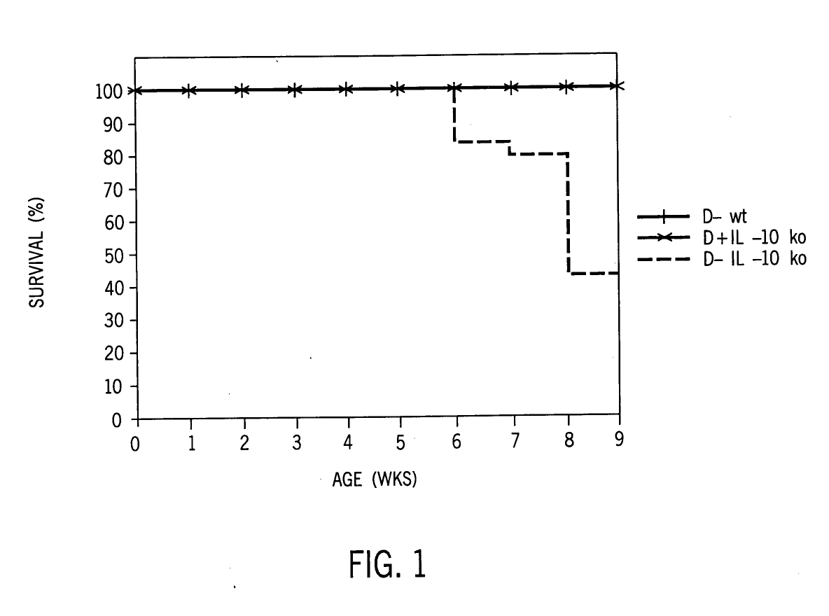

STATISTICAL ANALYSIS. Experiments were repeated as necessary and where possible, values were reported as the means from multiple experiments. A two-sample test for binomial proportions was used for statistical analysis of the percentage values shown in FIG. 1 as described (Rosner 1986). Body weights and weight gains were analyzed by repeated measures ANOVA using simple contrasts to compare diet groups (main effects). Data were subjected to two-way ANOVA using diet and IL-10 genotype as the grouping factors. All post-hoc multiple comparisons were made using the Fishers protected Least Significant Difference test. Values were compared using a statistics program (Statview Student, Abacus Concepts, Berkeley, CA) for the Macintosh computer and values of P<0.05 were considered significant.

FIG. 1. Vitamin D deficiency induces the mortality of IL-10 KO mice. Vitamin D deficient IL-10 KO weanling mice were randomly split into 2 groups. One group was maintained vitamin D deficient (−D, n=26) and the other was fed the same diet which contained 5.0 μg cholecalciferol/d for the remainder of the experiment (+D, n=10). Vitamin D deficient WT (−D, n=20) mice were also used in these experiments. Vitamin D deficient IL-10 KO mice died following the development of diarrhea. Vitamin D deficient WT and vitamin D sufficient IL-10 KO mice did not develop diarrhea or die.

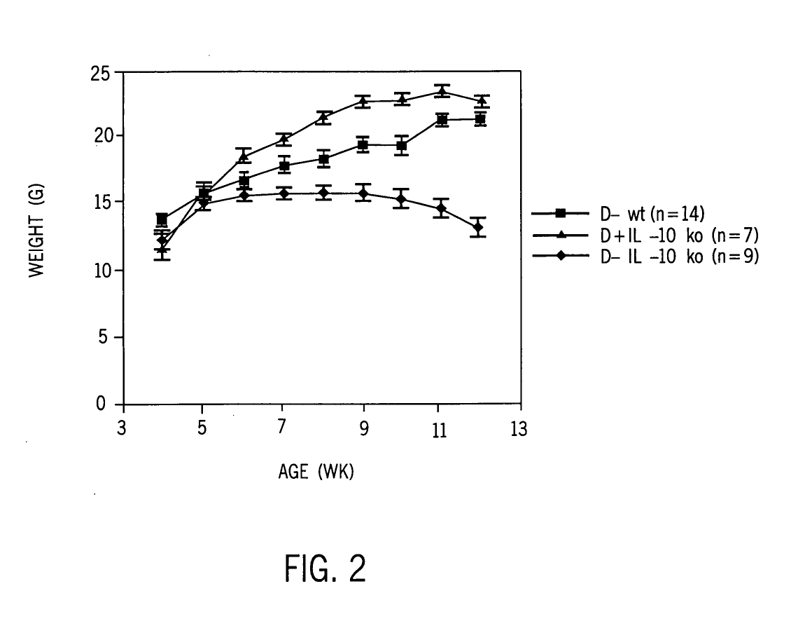

FIG. 2. Growth curves for vitamin D deficient and sufficient IL-10 KO mice and vitamin D deficient WT mice. Vitamin D deficient IL-10 KO weanling mice were randomly split into 2 groups. One group was maintained vitamin D deficient (−D, n=14 at the beginning of the experiment and n=5 at the end) and the other was fed the same diet which contained 5.0 μg cholecalciferol/d for the remainder of the experiment (+D, n=7). Vitamin D deficient WT (−D, n=9) mice were also used in these experiments. Vitamin D sufficient IL-10 KO mice grew rapidly compared to vitamin D deficient IL-10 KO mice. The growth of vitamin D deficient WT mice was retarded but constant over the 12 week period (+D WT mice weighed significantly less then +D IL-10 KO mice from 7 to 11 weeks of age, P<0.05) and by 12 weeks the vitamin D deficient WT mice matched the vitamin D sufficient mice in weight. Vitamin D deficient IL-10 KO mice (* weighed significantly less then +D IL-10 KO mice, P<0.05) stopped growing at 6 weeks of age and began to lose weight and undergo a severe wasting disease which eventually resulted in the death of the mice (n=5 by 12 weeks). The values are means ±SEM.

Results

MORTALITY OF VITAMIN D DEFICIENT IL-10 KO MICE. FIG. 1 shows that vitamin D deficient IL-10 KO mice begin to die at 7 weeks of age and that by 9 weeks of age 58% (15 dead of 26 total) of the vitamin D deficient IL-10 KO mice were dead. After 9 weeks of age vitamin D deficient IL-10 KO mice continued to waste and the death rate increased. In contrast, the vitamin D sufficient IL-10 KO (n=10) and the vitamin D deficient WT (n=20) mice appeared healthy even at 13 weeks of age.

The vitamin D deficient IL-10 KO mice were growth retarded compared to vitamin D sufficient IL-10 KO and vitamin D deficient WT mice (FIG. 2). The vitamin D deficient WT mice grew slower than the vitamin D sufficient IL-10 KO mice but by 12 weeks of age the vitamin D sufficient IL-10 KO and vitamin D deficient WT mice were the same size. By 6 weeks of age and thereafter the vitamin D deficient IL-10 KO mice had stopped growing and were significantly smaller than the vitamin D deficient WT mice (FIG. 2). At 9 weeks of age vitamin D deficient IL-10 KO mice began to eat less and rapidly lost additional weight over the next 3 weeks. Subsequent experiments were terminated at 9 weeks to prevent unnecessary pain and suffering of the IL-10 KO mice. The vitamin D deficient IL-10 KO mice died following a wasting disease which was preceded by diarrhea.

IBD SYMPTOMS IN VITAMIN D DEFICIENT AND 1,25-DIHYDROXYCHOLECALCIFEROL SUPPLEMENTED IL-10 KO MICE. Vitamin D deficient WT and IL-10 KO mice weighed less than their 1,25(OH)₂D₃ supplemented counterparts at 9 weeks of age (Table 1). The weights of the vitamin D deficient IL-10 KO mice were lower than in previous experiments (FIG. 2); although in this case consistent with the accelerated weight loss observed previously in vitamin D deficient IL-10 KO mice. As expected the serum calcium values in 1,25(OH)₂D₃ supplemented mice were significantly (P<0.05) higher than the vitamin D deficient mice (Table 1). Hemoglobin levels and erythrocyte numbers were normal and not different in vitamin D deficient, vitamin D sufficient, and 1,25(OH)₂D₃ supplemented IL-10 KO and WT mice (data not shown).

WT mice that were vitamin D deficient and sufficient showed no signs of inflammation or abnormalities in the SI. Vitamin D deficient IL-10 KO mice had significantly more inflammation in the SI than their 1,25(OH)₂D₃ supplemented or vitamin D sufficient counterparts (P<0.05, Table 1 and data not shown). Although the vitamin D deficient IL-10 KO mice were the smallest in size, necropsy showed that they had extremely large SI.

SHORT TERM 1,25-DIHYDROXYCHOLECALCIFEROL TREATMENT AND IBD SEVERITY. There were no significant differences in the weight of any of the mice following 2 week 1,25-dihydroxycholecalciferol treatment (data not shown). The SI of the vitamin D deficient IL-10 KO mice however were enlarged and weighed significantly more (P<0.05) than the SI from 1,25(OH)₂D₃ supplemented IL-10 KO, vitamin D deficient WT and 1,25(OH)₂D₃ supplemented WT mice (Table 2). In fact the SI from vitamin D deficient IL-10 KO mice were 9.9% of the total body weight which is 2-fold higher than normal (about 5%, Table 2). 1,25(OH)₂D₃ treatment for as little as 2 weeks reduced the inflammation in the small intestine of IL-10 KO mice.

FOOD RESTRICTION VERSUS VITAMIN D DEFICIENCY AND THE SYMPTOMS OF IBD. In order to rule out the possibility that weight loss and not vitamin D deficiency was associated with the increased symptoms of IBD observed, the food intake of vitamin D sufficient IL-10 KO and WT mice was restricted (Table 3). Food restriction successfully decreased the weight of vitamin D sufficient IL-10 KO and WT mice, however the vitamin D deficient IL-10 KO mice were still significantly smaller (P<0.05, Table 3). The IL-10 KO mice were extremely ill by 9 weeks in this series of experiments and had already undergone severe wasting. Food restriction did not change the symptoms of IBD in the vitamin D sufficient mice. Food restricted vitamin D sufficient IL-10 KO mice did not develop overt enterocolitis or death which occurred in vitamin D deficient IL-10 KO mice. The SI of vitamin D sufficient food restricted IL-10 KO mice were not different than in previous experiments or compared to WT controls (Table 3). Histopathology confirmed the weight measurements in Table 3 (data not shown). The early symptoms of IBD in vitamin D deficient IL-10 KO mice were associated with vitamin D deficiency and not to a reduction in energy or food intake.

TABLE 1
HISTOPATHOLOGY OF VITAMIN D-SUFFICIENT
AND-DEFICIENT IL-10 KO AND WT MICE.

| | | | | Serum | |
|---|---|---|---|---|---|
| | | Vitamin D | | Calcium | Histology |
| Genotype | n | Status¹ | Weight (g) | mmol/L | Score |
| IL-10 KO² | 7 | −D | 12.4 ± 2.3* | 1.74 ± 0.28* | 3.0 ± 0.2* |
| IL-10 KO | 7 | 1,25(OH)₂D₃ | 20.9 ± 0.8 | 3.00 ± 0.30 | 1.7 ± 0.4 |
| WT | 4 | −D | 16.7 ± 1.9* | 1.67 ± 0.32* | 0 |
| WT | 4 | 1,25(OH)₂D₃ | 21.1 ± 1.0 | 2.72 ± 0.25 | 0 |
| ¹All of the mice were vitamin D deficient (−D) for the first 5 weeks of life. At 5 weeks the mice were divided into 2 groups: half were supplemented with | | | | | |
| # 0.005 μg/day 1,25(OH)₂D₃ for 4 weeks. Values are means ± SEM. | | | | | |

*significantly different from supplemental counterpart, P < 0.05.
²IL, interleukin: KO, knockout: −D, vitamin D deficient: 1,25(OH)₂D₃, 1,25-dihydroxycholecalciferol: WT, wildtype.

TABLE 2
1,25(OH)₂D₃ TREATMENT DECREASES
ENTEROCOLITIS IN IL-10 KO MICE.

| | | | Small Intestine | |
|---|---|---|---|---|
| Genotype | n | Vitamin D Status¹ | g | g/100 g body |
| IL-10 KO² | 8 | −D | 1.67 ± 0.04* | 9.9 ± 0.5* |
| IL-10 KO | 8 | 1,25(OH)₂D₃ | 1.08 ± 0.05 | 5.4 ± 0.3 |
| WT | 12 | −D | 0.97 ± 0.02 | 5.8 ± 0.2 |
| WT | 8 | 1,25(OH)₂D₃ | 1.06 ± 0.04 | 5.3 ± 0.2 |
| ¹All of the mice were vitamin D deficient (−D) for the first 7 weeks of life. At 7 weeks of age, the vitamin D-deficient IL-10 KO mice begin to show symptoms of | | | | |
| # enterocolities (diarrhea and weight loss). The 7-week-old IL-10 KO and WT mice were divided into two group; half were supplemented with 0.2 μg/d 1,25(OH)₂D₃ for 2 weeks. Values are means ± SEM. | | | | |

*significantly greater than all other groups, P < 0.05.
²IL, interleukin: KO, knockout: −D, vitamin D deficient: 1,25(OH)₂D₃, 1,25-dihydroxycholecalciferol: WT, wildtype.

TABLE 3
VITAMIN D DEFICIENCY AND NOT A REDUCTION IN
FOOD INTAKE CAUSES INFLAMMATION IN THE
SMALL INTESTINE IN IL-10 KO AND WT MICE

| | | | | Small Intestine | |
|---|---|---|---|---|---|
| | | Vitamin D | | | g/100 g |
| Genotype | n | Status¹ | Weight, g | g | body |
| IL-10 KO | 7 | −D | 11.5 ± 1.2* | 1.79 ± 0.02* | 9.9 ± 0.4* |
| IL-10 KO | 4 | +D restricted | 16.1 ± 1.1 | 1.00 ± 0.02 | 6.2 ± 0.3 |
| WT | 4 | +D restricted | 15.9 ± 1.1 | 0.85 ± 0.06 | 6.7 ± 0.2 |
| ¹Five week old vitamin D deficient mice were divided into groups and either continued on diets that contained no added vitamin D (−D) or changed to a diet | | | | | |
| # that contained 5.0 μg/d (+D) cholecalciferol for 4 weeks. The +D mice were restricted in their food intake to only the amount eaten by the vitamin D deficient IL-10 KO mice | | | | | |
| # in the previous 24 h. Values are means ± SEM. | | | | | |

*significantly different from the other groups, P < 0.05.
²IL, interleukin: KO, knockout: −D, vitamin D deficient: +D, 1,25(OH)₂D₃ sufficient: WT, wildtype.

Discussion

Vitamin D deficiency exacerbates the symptoms of enterocolitis in IL-10 KO mice, and 1,25-dihydroxycholecalciferol treatment for as little as 2 weeks ameliorated IBD symptoms in these mice. These findings provide strong evidence that vitamin D status is an important factor in determining the incidence of IBD and furthermore establishes vitamin D as a physiological regulator of IBD. This is the first experimental evidence, which shows a link between vitamin D status and IBD.

The time course of IBD development in vitamin D deficient IL-10 KO mice is comparable to IBD which develops in IL-10 KO mice housed in conventional animal facilities (Kuhn et al. 1993). It is possible (although unlikely) that the microflora in the GI tract of IL-10 KO mice is disturbed during vitamin D deficiency such that disease causing microbes expand and multiply to cause disease. Experiments to test this possibility could be done in vitamin D deficient germfree mice, although in the absence of any microflora enterocolitis would probably not develop. It is more likely that the microflora does not change in response to vitamin D status but instead the absence of vitamin D changes the immune response and the result for IL-10 KO mice is more severe IBD.

Accumulating evidence suggests that vitamin D is a regulator of CD4+ T cells, which cause autoimmune disease (Cantorna et al. 1996, Cantorna et al. 1998c). One possible mechanism of vitamin D action is in the negative regulation of CD4+ T cells, which cause IBD. Vitamin D has been shown to directly inhibit the effector functions of CD4+ T cells both in vitro and in vivo (Cippitelli and Santoni 1998, Lemire 1992). The other possibility is that vitamin D is a positive regulator of T cells or other cells which inhibit the induction or function of IBD causing T cells. Two possible vitamin D targets are transforming growth factor (TGF)-β1 and IL-4 secreting cells (Cantorna et al. 1998c). Increased production of TGF-β1 and IL-4 have been shown to occur in mice treated with 1,25-dihydroxycholecalciferol in vivo (Cantorna et al. 1998c). Furthermore, the production of TGF-β1 and IL-4 is associated with the inhibition of T cell effector function and suppression of many autoimmune diseases (Groux et al. 1997). Vitamin D regulation of the immune system is probably complex and includes multiple targets, which together explain the mechanism by which 1,25-dihydroxycholecalciferol suppresses the development of IBD.

Standard treatments of patients with IBD include short-term high dose and long term low dose prednisone use (Andreassen et al. 1998, Podolosky 1991). Prednisone and other corticosteroid therapies result in a decreased bone mineral density and many times result in higher risks for vertebral fracture (Andreassen et al. 1997, Andreassen et al. 1998). Vitamin D supplementation of patients on corticosteroids has been shown to prevent steroid induced bone loss (Buckley et al. 1996). The hormonally active form of vitamin D (1,25-dihydroxycholecalciferol) is known to increase bone mineralization when given to experimental animals (Cantorna et al. 1998b) and people (Ongphiphadhanakul et al. 2000). Thus a further benefit of vitamin D and or 1,25-dihydroxycholecalciferol supplementation may be the maintenance of bone mineral density.

The data suggest that 1,25-dihydroxycholecalciferol and its analogs are novel and effective treatments for IBD patients. A possible limitation of 1,25-dihydroxycholecalciferol treatment is the hypercalcemia, which can result. The greatest promise thus may be for vitamin D analogs used in combination with the standard treatments. The standard treatments often work well but have many side effects; like bone loss which vitamin D analogs could reverse or block entirely. Vitamin D analogs in combination with corticosteroids, or sulfasalazine drugs could reduce the effective dose of these drugs, limit side effects and prove to be novel and effective treatments for human IBD.

For treatment purposes, the novel compounds of this invention defined by formula I may be formulated for pharmaceutical applications as a solution in innocuous solvents, or as an emulsion, suspension or dispersion in suitable solvents or carriers, or as pills, tablets or capsules, together with solid carriers, according to conventional methods known in the art. Any such formulations may also contain other pharmaceutically-acceptable and non-toxic excipients such as stabilizers, anti-oxidants, binders, coloring agents or emulsifying or taste-modifying agents.

The compounds may be administered orally, topically, parenterally or transdermally. The compounds are advantageously administered by injection or by intravenous infusion or suitable sterile solutions, or in the form of liquid or solid doses via the alimentary canal, or in the form of creams, ointments, patches, or similar vehicles suitable for transdermal applications. Doses of from 0.01 μg to 100 μg per day of the compounds are appropriate for treatment purposes, such doses being adjusted according to the activity of the particular compound being used, the disease to be treated, its severity and the response of the subject as is well understood in the art. Each compound may be suitably administered alone, or together with graded doses of another active vitamin D compound—e.g. 1α-hydroxyvitamin D₂ and/or D₃, in combination with 1α,25-dihydroxyvitamin D₃—in situations where different degrees of bone mineral mobilization and calcium transport stimulation is found to be advantageous.

Compositions for use in the above-mentioned treatment of IBD comprise an effective amount of one or more vitamin D compound as defined by the above formula I as the active ingredient, and a suitable carrier. An effective amount of such compounds for use in accordance with this invention is from about 0.01 μg to about 100 μg per gm of composition, and may be administered topically, transdermally, orally or parenterally in dosages of from about 0.01 μg/day to about 100 μg/day.

The compounds may be formulated as creams, lotions, ointments, topical patches, pills, capsules or tablets, or in liquid form as solutions, emulsions, dispersions, or suspensions in pharmaceutically innocuous and acceptable solvent or oils, and such preparations may contain in addition other pharmaceutically innocuous or beneficial components, such as stabilizers, antioxidants, emulsifiers, coloring agents, binders or taste-modifying agents.

The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof.

Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion.

Formulations for rectal administration may be in the form of a suppository incorporating the active ingredient and carrier such as cocoa butter, or in the form of an enema.

Formulations suitable for parenteral administration conveniently comprise a sterile oily or aqueous preparation of the active ingredient which is preferably isotonic with the blood of the recipient.

Formulations suitable for topical administration include liquid or semi-liquid preparations such as liniments, lotions, applicants, oil-in-water or water-in-oil emulsions such as creams, ointments or pastes; or solutions or suspensions such as drops; or as sprays.

The formulations may conveniently be presented in dosage unit form and may be prepared by any of the methods well known in the art of pharmacy. By the term “dosage unit” is meant a unitary, i.e. a single dose which is capable of being administered to a patient as a physically and chemically stable unit dose comprising either the active ingredient as such or a mixture of it with solid or liquid pharmaceutical diluents or carriers.

1-27. (Canceled)

28. A method of treating inflammatory bowel disease comprising administering to a patient with said disease an effective amount of a vitamin D compound having the formula: 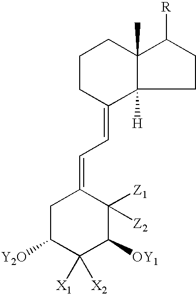
where Y₁ and Y₂, which may be the same or different, are each selected from the group consisting of hydrogen and a hydroxy-protecting group, where Z₁ and Z₂ are both hydrogen or Z₁ and Z₂ together are ═CH₂, where X, and X₂ are both hydrogen, or one is hydrogen and the other is O-aryl, O-alkyl, alkyl, hydroxyalkyl or fluoroalkyl and can have an α or β configuration, or taken together represent an alkylidene group ═CR₆R₇ where R₆ and R₇, which may be the same or different, are each selected from hydrogen, alkyl, hydroxyalkyl and fluoroalkyl, or, when taken together represent the group —(CH₂)ₓ— where x is an integer from 2 to 5, and can have an α or β configuration, and where the group R is represented by the structure: 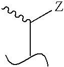
where the stereochemical center at carbon 20 may have the R or S configuration, and where Z is selected from Y, —OY, —CH₂OY, —C≡CY and —CH═CHY, where the double bond may have the cis or trans geometry, and where Y is selected from hydrogen, methyl, —COR⁵ and a radical of the structure: 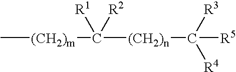
where m and n, independently, represent the integers from 0 to 5, where R¹ is selected from hydrogen, deuterium, hydroxy, protected hydroxy, fluoro, trifluoromethyl, and C₁₋₅-alkyl, which may be straight chain or branched and, optionally, bear a hydroxy or protected-hydroxy substituent, and where each of R², R³, and R⁴, independently, is selected from deuterium, deuteroalkyl, hydrogen, fluoro, trifluoromethyl and C₁₋₅ alkyl, which may be straight-chain or branched, and optionally, bear a hydroxy or protected-hydroxy substituent, and where R¹ and R², taken together, represent an oxo group, or an alkylidene group, ═CR²R³, or the group —(CH₂)ₚ—, where p is an integer from 2 to 5, and where R³ and R⁴, taken together, represent an oxo group, or the group —(CH₂)_{q}—, where q is an integer from 2 to 5, and where R⁵ represents hydrogen, hydroxy, protected hydroxy, or C₁₋₅ alkyl and wherein any of the CH-groups at positions 20, 22, or 23 in the side chain may be replaced by a nitrogen atom, or where any of the groups —CH(CH₃)—, —CH(R³)—, or —CH(R²)— at positions 20, 22, and 23, respectively, may be replaced by an oxygen or sulfur atom.

29. The method of claim 28 where the vitamin D compound is a 1α-hydroxy compound.

30. The method of claim 28 where the vitamin D compound is 1α, 25-dihydroxyvitamin D₃.

31. The method of claim 28 where the disease is ulcerative colitis.

32. The method of claim 28 where the disease is Crohn's disease.

33. The method of claim 28 wherein the compound is administered orally.

34. The method of claim 28 wherein the compound is administered parenterally.

35. The method of claim 28 wherein the compound is administered transdermally.

36. The method of claim 28 wherein the compound is administered in a dosage of from 0.01 μg to 100 μg per day.

37. The method of claim 28 wherein the compound is selected from the group consisting of 1,25-dihydroxyvitamin D₃; 1α-hydroxyvitamin D₃; 1,25-dihydroxyvitamin D₂; 19-nor-1,25-dihydroxyvitamin D₂; 26,27-hexafluoro-1,25-dihydroxyvitamin D₂; 1,25-dihydroxy-24(E)-dehydro-24-homo-vitamin D₃; 19-nor-1,25-dihydroxy-21-epi-vitamin D₃; 1α,25 dihydroxyvitamin D₃ triacetate; and 25-acetyl-1α,25-dihydroxyvitamin D₃.

38. The method of claim 28 wherein the patient is on a low calcium diet.

39. A method of preventing development of or delaying onset of inflammatory bowel disease in susceptible individuals comprising administering to the individual an effective amount of a vitamin D compound having the formula: 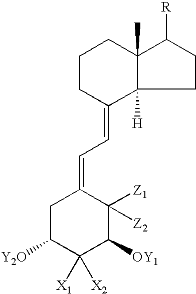
where Y₁ and Y₂, which may be the same or different, are each selected from the group consisting of hydrogen and a hydroxy-protecting group, where Z₁ and Z₂ are both hydrogen or Z₁ and Z₂ together are ═CH₂, where X₁ and X₂ are both hydrogen, or one is hydrogen and the other is O-aryl, O-alkyl, alkyl, hydroxyalkyl or fluoroalkyl and can have an α or β configuration, or taken together represent an alkylidene group ═CR₆R₇ where R₆ and R₇, which may be the same or different, are each selected from hydrogen, alkyl, hydroxyalkyl and fluoroalkyl, or, when taken together represent the group —(CH₂)ₓ— where x is an integer from 2 to 5, and can have an α or β configuration, and where the group R is represented by the structure: 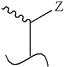
where the stereochemical center at carbon 20 may have the R or S configuration, and where Z is selected from Y, —OY, —CH₂OY, —C≡CY and —CH═CHY, where the double bond may have the cis or trans geometry, and where Y is selected from hydrogen, methyl, —COR⁵ and a radical of the structure: 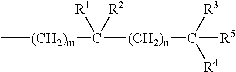
where m and n, independently, represent the integers from 0 to 5, where R¹ is selected from hydrogen, deuterium, hydroxy, protected hydroxy, fluoro, trifluoromethyl, and C₁₋₅-alkyl, which may be straight chain or branched and, optionally, bear a hydroxy or protected-hydroxy substituent, and where each of R², R³, and R⁴, independently, is selected from deuterium, deuteroalkyl, hydrogen, fluoro, trifluoromethyl and C₁₋₅ alkyl, which may be straight-chain or branched, and optionally, bear a hydroxy or protected-hydroxy substituent, and where R¹ and R², taken together, represent an oxo group, or an alkylidene group, ═CR²R³, or the group —(CH₂)ₚ—, where p is an integer from 2 to 5, and where R³ and R⁴, taken together, represent an oxo group, or the group —(CH₂)_{q}—, where q is an integer from 2 to 5, and where R⁵ represents hydrogen, hydroxy, protected hydroxy, or C₁₋₅ alkyl and wherein any of the CH-groups at positions 20, 22, or 23 in the side chain may be replaced by a nitrogen atom, or where any of the groups —CH(CH₃)—, —CH(R³)—, or —CH(R²)— at positions 20, 22, and 23, respectively, may be replaced by an oxygen or sulfur atom.

40. The method of claim 39 wherein the compound is 1α,25-dihydroxyvitamin D₃.

41. The method of claim 39 wherein the compound is a 1α-hydroxy compound.

42. The method of claim 39 wherein the compound is selected from the group consisting of 1,25-dihydroxyvitamin D₃; 1α-hydroxyvitamin D₃; 1,25-dihydroxyvitamin D₂; 19-nor-1,25-dihydroxyvitamin D₂; 26,27-hexafluoro-1,25-dihydroxyvitamin D₂; 1,25-dihydroxy-24(E)-dehydro-24-homo-vitamin D₃; 19-nor-1,25-dihydroxy-21-epi-vitamin D₃; 1α,25-dihydroxyvitamin D₃ triacetate; and 25-acetyl-1α,25-dihydroxyvitamin D₃.

43. The method of claim 39 wherein said effective amount comprises about 0.01 μg/day to about 100 μg/day of said compound.

44. The method of claim 39 wherein the compound is administered orally.

45. The method of claim 39 wherein the compound is administered parenterally.

46. The method of claim 39 wherein the compound is administered transdermally.

47. The method of claim 39 wherein the disease is ulcerative colitis.

48. The method of claim 39 wherein the disease is Crohn's disease.

49. The method of claim 39 wherein the individual is on a low calcium diet.
